(19) United States
(12) Patent Application Publication
(10) Pub. No.: US 2003/0082225 A1
(43) Pub. Date: May 1, 2003

(54) Sterile, breathable patch for treating wound pain

(76) Inventor: Paul Arthur Mason, Flemington, NJ (US)

Correspondence Address: PENNIE AND EDMONDS, 1155 AVENUE OF THE AMERICAS, NEW YORK, NY, 100362711

(21) Appl. No.: 10/045,730

(22) Filed: Oct. 19, 2001

(51) Int. Cl. A61K 31/485; A61K 9/70; A61K 31/137

(52) U.S. Cl. 424/449; 514/282; 514/650

(57) ABSTRACT

An intradermal patch having a permeable backing coated with a polyvinylpyrrolidone-based hydrogel and containing one or more local anesthetics. The patch is breathable, non-irritating upon application and removal, soothing, and sterile. The patch is useful for treating the pain associated with non-intact skin indications.

### I. FIELD OF THE INVENTION

The present invention relates to breathable patches for topically delivering local anesthetics to treat or prevent pain.

### II. BACKGROUND

Pain results from the noxious stimulation of nerve endings. Nociceptive pain is caused by noxious stimulation of nociceptors, which then transmit impulses over intact neural pathways to the spinal neurons and then to the brain. GOODMAN & GILMAN'S THE PHARMACOLOGICAL BASIS OF THERAPEUTICS 529 (Joel G. Hardman et al. eds., 9th ed. 1996); HARRISON'S PRINCIPLES OF INTERNAL MEDICINE 53-58 (Anthony S. Fauci et al. eds., 14th ed. 1998).

In contrast to pain treatment with systemic agents, pain can be treated locally by topically administering a local anesthetic directly to the painful area to block the nociceptive mechanistic pathway. Local anesthetics prevent the generation and conduction of nociceptive nerve impulses. Thus, for example, a local anesthetic can be injected intradermally (non-systemic injection within the skin), applied to an open wound or bum, or topically applied to intact skin. Advantages of topical local-anesthetic administration over systemic administration of pain relievers include decrease or preclusion of side effects, improved patient compliance, and reversible action (i.e., the action can be reversed by removing the anesthetic from the application site). TRANSDERMAL AND TOPICAL DRUG DELIVERY SYSTEMS 33-112 (Tapash K. Ghosh et al eds., 1997).

A variety of drug classes have local-anesthetic properties and can be administered topically. Traditional local anesthetics or sodium-channel blockers, such as lidocaine prevent the generation and conduction of nerve impulses by decreasing or preventing the large transient increase in the permeability of excitable membranes to Na+. Other agents with local-anesthetic properties include analgesics, such as non-steroidal anti-inflammatories (“NSAIDs”), see, for example, TRANSDERMAL AND TOPICAL DRUG DELIVERY SYSTEMS 87-93 (Tapash K. Ghosh et al. eds., 1997) and opioids, such as morphine. See e.g., U.S. Pat. No. 5,948,389 (issued Sep. 7, 1999); Christoph Stein & Alexander Yassouridis 71 Pain 119 (1997).

N-methyl-D-aspartate (“NMDA”) receptor antagonists, such as ketamine also have local-anesthetic properties and topical administration is as an effective neuropathic pain treatment. See, for example, U.S. Pat. No. 5,817,699 (issued Oct. 6, 1998). In another example, topical administration of antidepressant medications, such as amitriptyline, has been reported effective for neuropathic pain treatment. See, for example, U.S. Pat. No. 6,211,171 (issued Apr. 3, 2001); J. Sawynok et al., 82 PAIN 149 (1999). In addition, topical administration of a combination of a tricyclic antidepressant and an NMDA-receptor antagonist is reported to have excellent local-anesthetic properties when topically applied and is useful for treatment of neuropathic pain, U.S. Pat. No. 6,197,830 (issued Mar. 6, 2001).

Patch-type delivery systems are often used to deliver local anesthetics to intact skin. In general, these patches comprise a backing that is impermeable to air and moisture (not breathable). A patch containing a local anesthetic has advantages over simple topical application. One advantage is that the dose is better regulated. Other advantages of patches are constant rate of delivery, longer duration of action (the ability of to adhere to the skin for 1, 3, 7 days or longer), improved patient compliance, non-invasive dosing, and reversible action (i.e., the patch can simply be removed). Hydrogels have been used in conjunction with patches on intact skin to deliver pharmaceuticals. For example, U.S. Pat. No. 6,096,334 (issued Aug. 1, 2000) describes adhesive hydrogel patches for applying medication to intact skin. Advantageously, hydrogels are sterilizable, air permeable, promote hydration, and provide a soothing and cooling effect. Ming-Hong et al. 11 NUCLEAR SCIENCE AND TECHNIQUES 72 (2000); Yoshii et al. 55 RADIATION PHYSICS AND CHEMISTRY 133 (1999).

Generally, the above-described hydrogels have not been used in conjunction with patches to treat non-intact skin indications, such as open wounds and bums because of the difficulty to package such hydrogel patches with breathable backings in a sterile environment. Open wounds and bums require breathable and sterile patches to prevent infection. In fact, generally patches have not been used to deliver local anesthetics to wounds and bums because of the difficulty associated with packaging breathable, non-irritating, soothing patches in a sterile environment.

Thus, there is a need for breathable, sterile, non-irritating, and soothing patches that can topically deliver local anesthetics to treat the pain associated with non-intact-skin indications, such as wounds and bums.

Citation or identification of any reference in the Background section of this application is not an admission that such reference is prior art to the present invention.

### III. SUMMARY

In one embodiment, the invention is directed to polyvinylpyrrolidone-based hydrogel patches comprising a local anesthetic and having a breathable backing, which are useful for treating the pain associated with non-intact skin indications. Breathability is essential to prevent infection. And because the patches of the invention are hydrogel based, they provide a soothing and cooling effect when topically applied and will not further irritate the wound upon removal. Furthermore, the patches of the invention are stabile to γ-radiation sterilization, thus, can be sterilized after packaging. Because the patches are soothing, non-irritating, breathable, and packaged in a sterile environment, they can be distributed for treating the pain associated with non-intact skin indications.

In another embodiment, the invention is directed to a patch comprising a breathable backing coated with a polyvinylpyrrolidone-based hydrogel, the hydrogel comprising one or more local anesthetics or a pharmaceutically acceptable salt thereof.

In yet another embodiment, the invention is directed to a package containing a sterile patch, the patch comprising a breathable backing coated with a polyvinylpyrrolidone-based hydrogel, the hydrogel comprising one or more local anesthetics or a pharmaceutically acceptable salt thereof.

In still another embodiment, the invention concerns a method of inducing local anesthesia in a mammal comprising topically applying a patch to the mammal, the patch comprising a breathable backing coated with a polyvinylpyrrolidone-based hydrogel, the hydrogel comprising one or more local anesthetics or a pharmaceutically acceptable salt thereof.

In one more embodiment, the invention provides a method of treating the pain associated with a non-intact skin indication in a mammal comprising topically applying a sterile patch to the non-intact skin indication, the patch comprising a breathable backing coated with a polyvinylpyrrolidone-based hydrogel, the hydrogel comprising one or more local anesthetics or a pharmaceutically acceptable salt thereof.

The present invention may be understood more fully by reference to the following detailed description and illustrative examples, which are intended to exemplify non-limiting embodiments of the invention.

### IV. DEFINITIONS

As used herein, a “patch of the invention” means an intradermal delivery patch comprising a breathable backing coated with a polyvinylpyrrolidone-based hydrogel, the hydrogel comprising one or more local anesthetics or a pharmaceutically acceptable salt thereof.

As used herein, the phrase “pre-hydrogel mixture” means a homogeneous mixture comprising:

(a) from about 5% to about 35% by weight, preferably, from about 10% to about 30%, more preferably, from about 15% to about 20% by weight of polyvinylpyrrolidone having an average molecular weight ranging from about 900,000 to about 1,500,000 Daltons;

(b) from about 0.5% to about 20% by weight of a local anesthetic, preferably from about 2% to about 10%; and

(c) the remainder water.

which mixture, when subjected to high-energy radiation, such as electron-beam radiation, forms a hydrogel.

As used herein, the term “wound” refers broadly to injuries to the skin and subcutaneous tissue. Wounds may be classified into one of four grades depending on the depth of the wound: Grade I: wounds limited to the epithelium; Grade II: wounds extending into the dermis; Grade II: wounds extending into the subcutaneous tissue; and Grade IV (or full-thickness wounds): wounds wherein bones are exposed. The term “wound” further includes infected wounds, chronic wounds, incurable wounds, and surgically closed wounds. The term “wound” also encompasses burns, such as chemical, radiation, and thermal burns; pressure sores; venous stasis ulcers; and diabetic ulcers. The patches of the invention can be used to treat the pain associated with all wound types.

As used herein, the phrase “non-intact skin indication” means broken, cut, punctured, or otherwise traumatized skin or areas on the body where the skin has been compromised. Non-intact skin indications include wounds and bums. The patches of the invention can be used to treat the pain associated with non-intact skin indications.

As used herein, a “therapeutically effective amount” of a local anesthetic means the amount of the local anesthetic required in a topical, intradermal patch of the invention to induce a local-anesthetic effect sufficient to treat or ameliorate pain in a mammal.

As used herein, the term mammal means any mammal, for example, but not limited to humans; pets, such as dogs and cats; farm mammals, such as horses, cows, pigs, and sheep; and laboratory animals, such as monkeys, guinea pigs, rats, and mice. Preferably, a “mammal” is a human.

As used herein, the term “intradermal administration” means administration of a pharmaceutical to the skin of a mammal, preferably a human, to deliver the pharmaceutical to the local tissue under and around the site of administration. Preferably, intradermal administration is effected without significant absorption of the pharmaceutical into the mammal's blood stream. The purpose of intradermal administration is to elicit a local affect in contrast to transdermal administration where the objective is to transfer the pharmaceutical through the skin and into the blood stream for a systemic effect.

As used herein, the phrases “topical administration” and “topical delivery” of a pharmaceutical (e.g., a local anesthetic) means intradermal administration of the pharmaceutical by topical application of the pharmaceutical or a patch or composition comprising the pharmaceutical. For example, applying a patch of the invention to a non-intact-skin indication, such as a wound or burn. The term “topical composition” means a pharmaceutical composition designed for topical administration and containing a pharmaceutical.

As used herein, the phrase “intradermally acceptable” means any pharmaceutical, excipient or other component of a topical formulation that is safe or approved for intradermal or topical administration in mammals.

The phrase “pharmaceutically acceptable salt(s),” as used herein includes, but is not limited to, salts of acidic or basic groups that may be present in the compounds of the invention. Compounds of the invention that are basic in nature are capable of forming a wide variety of salts with various inorganic and organic acids. The acids that may be used to prepare pharmaceutically acceptable salts of such basic compounds are those that form salts comprising pharmacologically acceptable anions including, but not limited to, acetate, benzenesulfonate, benzoate, bicarbonate, bitartrate, bromide, calcium edetate, camsylate, carbonate, chloride, bromide, iodide, citrate, dihydrochloride, edetate, edisylate, estolate, esylate, fumarate, gluceptate, gluconate, glutamate, glycollylarsanilate, hexylresorcinate, hydrabamine, hydroxynaphthoate, isethionate, lactate, lactobionate, malate, maleate, mandelate, mesylate, methylsulfate, muscate, napsylate, nitrate, panthothenate, phosphate/diphosphate, polygalacturonate, salicylate, stearate, succinate, sulfate, tannate, tartrate, teoclate, triethiodide, and pamoate (i.e., 1,1′-methylene-bis-(2-hydroxy-3-naphthoate)). Compounds of the invention that include an amino moiety also can form pharmaceutically acceptable salts with various amino acids, in addition to the acids mentioned above. Compounds of the invention that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

As used herein, the term “solvate” means a compound of the invention or a salt thereof, that further includes a stoichiometric or non-stoichiometric amount of a solvent bound by non-covalent intermolecular forces. Preferred solvents are volatile, non-toxic, and/or acceptable for topical administration to humans.

As used herein, the term “hydrate” means a compound of the invention or a salt thereof, that further includes a stoichiometric or non-stoichiometric amount of water bound by non-covalent intermolecular forces.

The term “clathrate” means a compound of the invention or a salt thereof in the form of a crystal lattice that contains spaces (e.g., channels) that have a guest molecule (e.g., a solvent or water) trapped within.

The term “prodrug” refers to a compound that, following administration in a mammal, converts, via a biotransformation, into an antidepressant or an NMDA-receptor antagonist in vivo. Prodrugs can be synthesized using well-known methods, such as those described by 1 BURGER'S MEDICINAL CHEMISTRY AND DRUG DISCOVERY, 172-178, 949-982 (Manfred E. Wolff ed., 5th ed. 1995).

### V. DETAILED DESCRIPTION

The patches of the invention can be used to treat, prevent, or ameliorate the pain associated with non-intact skin indications, such as wounds and burns and other pain indications via topical application. The patches of the invention comprise a cross-linked polyvinylpyrrolidone hydrogel layer comprising a local anesthetic or mixture of local anesthetics and a breathable backing layer. Preferably, the patch is packaged and sterilized with γ-radiation but other sterilization means, such as ethylene oxide, may also be used.

A. Local Anesthetics

As used herein, the term “local anesthetic” means any compound or composition that provides local numbness or analgesia or any drug that provides a regional blockage of nociceptive pathways (afferent and/or efferent). The local anesthetic can be any local anesthetic known or to be developed.

In general, the local anesthetic will comprise from about 0.5% to about 20% by weight of the hydrogel portion of the patch, preferably, from about 1% to about 15%, more preferably from about 2% to about 10% by weight of the hydrogel portion of the patch.

Compounds with local-anesthetic properties can contain one or more chiral centers and/or double bonds and, therefore, exist as stereoisomers, such as double-bond isomers (i.e., geometric isomers), enantiomers, or diastereomers. As used herein, the term “local anesthetic” encompass all such enantiomers and stereoisomers, that is, both the stereomerically-pure form (e.g., geometrically pure, enantiomerically pure, or diastereomerically pure) and enantiomeric and stereoisomeric mixtures, e.g., racemates. The term “local anesthetic” further encompasses all pharmaceutically acceptable salts, all complexes (e.g., hydrates, solvates, and clathrates), and all prodrugs of NMDA-receptor antagonist.

1. Sodium-Channel Blockers as Local Anesthetics

Examples of local anesthetics suitable for use with the invention include sodium-channel blockers and pharmaceutically acceptable salts thereof. Sodium-channel blockers, such as lidocaine prevent the generation and conduction of nerve impulses by decreasing or preventing the large transient increase in the permeability of excitable membranes to Na+. Examples of sodium-channel blockers include, but are not limited to, ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutylp-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, or pharmaceutically-acceptable salts thereof, or mixtures thereof. Preferred sodium-channel blockers, include lidocaine, procaine, bupivacaine, prilocaine, mepivacaine, etidocaine, ropivacaine, dibucaine, and pharmaceutically-acceptable salts thereof and mixtures thereof. The most preferred local anesthetic is lidocaine and pharmaceutically acceptable salts thereof.

2. Opioids as Local Anesthetics

Opioids and pharmaceutically acceptable salts thereof, such as morphine are known to have local-anesthetic properties when topically administered in mammals. See, for example, U.S. Pat. No. 5,948,389 (issued Sep. 7, 1999) and Christoph Stein & Alexander Yassouridis 71 Pain 119 (1997).

As used herein the term “opioid” means all agonists and antagonists of opioid receptors, such as mu (μ), kappa (κ), and delta (δ) opioid receptors and subtypes thereof. For a discussion of opioid receptors and subtypes see GOODMAN & GILMAN'S THE PHARMACOLOGICAL BASIS OF THERAPEUTICS 521-525 (Joel G. Hardman et al. eds., 9th ed. 1996), hereby expressly incorporated herein by reference. The opioid can be any opioid receptor agonist or antagonist known or to be developed. Preferred opioids interact with the opioid receptor, the κ-opioid receptor, or both. Preferably, the opioid is an opioid-receptor agonist.

Examples of suitable opioids include, but are not limited to, alfentanil, allylprodine, alphaprodine, anileridine, benzylmorphine, benzitramide, nor-binaltorphimine, bremazocine, buprenorphine, butorphanol, clonitazene, codeine, CTOP, DAMGO, desomorphine, dextromoramide, dezocine, diampromide, dihydrocodeine, dihydrocodeine enol acetate, dihydromorphine, dimenoxadol, dimepheptanol, dimethylthiambutene, dioxaphetyl butyrate, dipipanone, diprenorphine, DPDPE, eptazocine, ethoheptazine, ethylketocyclazocine, ethylmethylthiambutene, etonitazene, etorphine, fentanyl, hydrocodone, hydromorphone, hydroxypethidine, isomethadone, ketobemidone, levorphanol, lofentanil, loperamide, meperidine, meptazinol, metazocaine, methadone, metopon, morphine, myrophine, nalbuphine, naltrindole, benzoylhydrazone, naltrexone, narceine, nicomorphine, norlevorphanol, normethadone, normorphine, norpipanone, opium, oxycodone, oxymorphone, papaveretum, papaverine, pentazocine, phenadoxone, phenazocine, phenoperidine, piminodine, pirtramide, proheptazine, promedol, propiram, propoxyphene, remifentanil, spiradoline, sufentanil, tilidine, U50,488, and U69,593, amiphenazole, cyclazocine, levallorphan, nalmefene, nalorphine, naloxone, and naltrexone or pharmaceutically-acceptable salts thereof, or mixtures thereof.

Examples of peptide opioids include, but are not limited to, Tyr-Gly-Gly-Phe-Leu ([Leu⁵]enkephalin), Tyr-Gly-Gly-Phe-Met ([Met⁵]enkephalin), Tyr-Gly-Gly-Phe-Leu-Arg-Arg-Ile-Arg-Pro-Lys-Leu-Lys-Trp-Asp-Asn-Gln (DynorphinA), Tyr-Gly-Gly-Phe-Leu-Arg-Arg-Gln-Phe-Lys-Val-Val-Thr (Dynorphin B), Tyr-Gly-Gly-Phe-Leu-Arg-Lys-Tyr-Pro-Lys (a-Neoendorphin), Tyr-Gly-Gly-Phe-Leu-Arg-Lsy-Tyr-Pro (β-Neoendorphin), Tyr-Gly-Gly-Phe-Met-Thr-Ser-Glu-Lys-Ser-Gln-Thr-Pro-Leu-Val-Thr-Leu-Phe-Lys-Asn-Ala-Ile-Ile-Lys-Asn-Ala-Tyr-Lys-Lys-Gly-Glu (βₕ-Endorphin), [D-Ala²,MePhe⁴Gly(ol)⁵]enkephalin (DAMGO), [D-Pen²,D-Pen⁵]enkephalin (DPDPE), [D-Ser2,Leu⁵]enkephalin-Thr⁶ (DSLET), [D-Ala²,D-Leu⁵]enkephalin (DADL), D-Phe-Cys-Tyr-D-Trp-Orn-Thr-Pen-Thr-NH₂(CTOP), [D-Ala²,N-MePhe⁴,Met(O)⁵-ol]enkephalin (FK-33824), Tyr-D-Ala-Phe-Asp-Val-Val-Gly-NH₂ ([D-Ala²]Deltorphin 1), Tyr-D-Ala-Phe-Glu-Val-Val-Gly-NH₂ ([D-Ala²Glu⁴]Deltorphin (Deltorphin II)), Tyr-Pro-Phe-Pro-NH² (Morphiceptin), Tyr-Pro-MePhe-D-Pro-NH² (PL-017), [D-Ala²,Leu⁵,Cys⁶]enkephalin (DALCE) or pharmaceutically-acceptable salts thereof, or mixtures thereof. Preferred opioids include morphine, loperamide, and loperamide derivatives such as those disclosed in U.S. Pat. Nos. 5,763,445; 5,981,513; 5,869,521; 5,744,458; 5,760,023; 5,798,093; 5,849,762; 5,811,078; 6,004,964; 5,962,477; 5,688,955; 5,888,494; 5,646,151; and 5,667,773 or pharmaceutically-acceptable salts thereof, or mixtures thereof, all of which patents are hereby expressly incorporated herein by reference. The most preferred opioid is morphine or a pharmaceutically-acceptable salt thereof.

3. Antidepressants as Local Anesthetics

Compounds administered orally to treat depression in mammals are also known to have local-anesthetic properties when administered intradermally and topically. As used herein the term “antidepressant” means any compound or composition known or to be discovered that, when tested according to standard in vivo or in vitro assays, displays receptor-binding properties or other mechanistic properties associated with the clinically approved antidepressants or any compound or composition known or to be discovered that has demonstrated clinical efficacy in treating depression in mammals including those compounds and compositions that have been approved for treating depression in humans. Classes of antidepressant agents include norepinephrine-reuptake inhibitors (NRIs”), selective-serotonin-reuptake inhibitors (SSRIs), monoamine-oxidase inhibitors (MAOIs), serotonin-and-noradrenaline-reuptake inhibitors (“SNRIs); corticotropin-releasing factor (CRF) antagonists, α-adrenoreceptor antagonists; NK1-receptor antagonists, 5-HTIA-receptor agonist, antagonists, and partial agonists, a typical antidepressants, and other antidepressants and pharmaceutically acceptable salts thereof.

An antidepressant can contain one or more chiral centers and/or double bonds and, therefore, exist as stereoisomers, such as double-bond isomers (i.e., geometric isomers), enantiomers, or diastereomers. As used herein, the term “antidepressant” encompass all such enantiomers and stereoisomers, that is, both the stereomerically-pure form (e.g., geometrically pure, enantiomerically pure, or diastereomerically pure) and enantiomeric and stereoisomeric mixtures, e.g., racemates. The term “antidepressant” further encompasses all pharmaceutically acceptable salts, all complexes (e.g., hydrates, solvates, and clathrates), and all prodrugs of antidepressants.

Notably, the intradermal patches of the invention involve topical administration, thus “antidepressants” unsuitable for systemic administration in mammals, because of toxicity or otherwise, may still be suitable for topical administration in combination with an NMDA-receptor antagonist according to the patches and methods of the invention. Antidepressants suitable for use in the invention can be identified by testing antidepressant compounds for local-anesthetic and peripheral antinociceptive properties according to standard pain models. See, for example, J. Sawynok et al., 82 PAIN 149 (1999); J. Sawynok et al., 80 PAIN 45 (1999), both of which citations are hereby expressly incorporated by reference herein.

Preferably an antidepressant is a norepinephrine-reuptake inhibitor, more preferably, a tricyclic antidepressant, most preferably, amitriptyline, even more preferably amitriptyline hydrochloride.

The term “antidepressant” as used herein includes compounds that when administered systemically in a mammal, inhibit norepinephrine-reuptake (“norepinephrine-reuptake inhibitors”) or that when tested according to standard in vivo or in vitro assays, display receptor-binding properties or other mechanistic properties associated with norepinephrine-reuptake inhibitors. One of skill in the art can readily identify norepinephrine-reuptake inhibitors by in vivo and in vitro assays. For example, norepinephrine-reuptake inhibitors can be identified by adapting the in vitro test method described by Wong et al, 61 J. PHARM. EXP. THERAP. 222 (1982); P. Skolnick et al., 86 BR. J. PHARMACOLOGY 637-644 (1985), hereby expressly incorporated herein by reference. Examples of norepinephrine-reuptake inhibitors include, but are not limited to amitriptyline, desmethylamitriptyline, clomipramine, doxepin, imipramine, imipramine-oxide, trimipramine; adinazolam, amiltriptylinoxide, amoxapine, desipramine, maprotiline, nortriptyline, protriptyline, amineptine, butriptyline, demexiptiline, dibenzepin, dimetacrine, dothiepin, fluacizine, iprindole, lofepramine, melitracen, metapramine, norclolipramine, noxiptilin, opipramol, perlapine, pizotyline, propizepine, quinupramine, reboxetine, tianeptine, and pharmaceutically acceptable salts thereof. Examples of other norepinephrine-reuptake inhibitors include the tricyclic compounds encompassed by the generic formula disclosed in U.S. Pat. No. 6,211,171 (issued Apr. 30, 2001) column 9, lines 30-65 and pharmaceutically acceptable salts thereof, hereby expressly incorporated herein by reference.

The term “antidepressants” also includes compounds that inhibit reuptake of serotonin (“serotonin reuptake inhibitors”) when systemically administered in mammals or that when tested according to standard in vivo or in vitro assays, display receptor-binding properties or other mechanistic properties associated with serotonin-reuptake inhibitors. One of skill in the art can readily identify serotonin-reuptake inhibitors. For example, serotonin-reuptake inhibitors can be identified by adapting the in vitro test methods described in Wong, et al., 8 NEUROPSYCHOPHARMACOLOGY 337 (1993); U.S. Pat. No. 6,225,324 (issued May 1, 2001), column 20, lines 20-67; and U.S. Pat. No. 5,648,396 (issued Jul. 15, 1997) column 15, line 33 through column 16, line 44, hereby expressly incorporated herein by reference. Examples of serotonin-reuptake inhibitors include, but are not limited to, binedaline, m-chloropiperzine, citalopram, duloxetine, etoperidone, femoxetine, fluoxetine, fluvoxamine, indalpine, indeloxazine, milnacipran, nefazodone, oxaflazone, paroxetine, prolintane, ritanserin, sertraline, tandospirone, venlafaxine and zimeldine, and pharmaceutically acceptable salts thereof.

The term “antidepressant” as used herein includes compounds that when administered systemically in a mammal, act as monoamine-oxidase inhibitors (“MAOIs”) or that when tested according to standard in vivo or in vitro assays, inhibit monoamine oxidase. One of skill in the art can readily identify MAOIs by in vivo and in vitro assays. For example, MAOIs can be identified by adapting the monoamine-oxidase inhibitory assay described in 12 Biochem. Pharmacol. 1439 (1963) and Kinemuchi et al., 35 J. NEUROCHEM. 109 (1980); U.S. Pat. No. 6,096,771 (issued Aug. 1, 2000), all of which citations are hereby expressly incorporated herein by reference.

Examples of non-selective MAO inhibitors include, but are not limited to, amiflamine, vanoxerine (boxeprazine), AGN 2253 (Nicholas Kiwi), iproniazid, isocarboxazid, M-3-PPC (Draxis), nialamid, phenelzine, pargyline, and tranylcypromine and pharmaceutically acceptable salts thereof.

Examples selective MAO A inhibitors include, but are not limited to, clorgyline, cimoxatone, befloxatone, brofaromine, bazinaprine, BW-616U (Burroughs Wellcome), BW-1370U87 (Burroughs Wellcome), CS-722 (RS-722) (Sankyo), E-2011 (Eisai), harmine, harmaline, moclobemide, PharmaProjects 3975 (Hoechst), RO 41-1049 (Roche), RS-8359 (Sankyo), T-794 (Tanabe Seiyaku), toloxatone, K-Y 1349 (Kalir and Youdim), LY-51641 (Lilly), LY-121768 (Lilly), M&B 9303 (May & Baker), MDL 72394 (Marion Merrell), MDL 72392 (Marion Merrell), sercloremine, and MO 1671 and pharmaceutically acceptable salts thereof.

Other MAO A inhibitors include budipine, caroxazone, D-1711 (Biocodex), fezolamine, FLA-334 (RAN-113) (Astra), FLA-289 (FLA-299, FLA-365, FLA-384, FLA-463, FLA-727) (Astra), K-11566 (Pharmacia Upjohn, Farmitalia), K-11829 (Pharmacia Upjohn, Farmitalia), metralindole, MPCPAM (Yissum), PharmaProjects 227 (Syntex/Roche), PharmaProjects 2806 (Fournier), PharmaProjects 1122, PharmaProjects 3311 (Roche), PharmaProjects 4433 (Roche), RS-2232 (Sankyo), and UP-614-04 (Bristol-Myers) and pharmaceutically acceptable salts thereof.

Other MAO inhibitors include bifemelane, brofaromide, hypericin, iproclozide, medifoxamine, nialamide, octamoxin, phenoxypropaazine, pivalyl benzhydrazine, prodipine, selegiline, and benmoxine and pharmaceutically acceptable salts thereof.

The term “antidepressant” as used herein includes compounds that when administered systemically in a mammal, act as serotonin- and noradrenaline-reuptake inhibitors (“SNRIs”) or that when tested according to standard in vivo or in vitro assays, display receptor-binding properties or other mechanistic properties associated with serotonin- and noradrenalin-reuptake inhibitors. One of skill in the art can readily identify SNRIs by in vivo and in vitro assays. For example, SNRIs can be identified by adapting the in vitro test method described in U.S. Pat. No. 6,172,097 (issued Jan. 9, 2001), hereby expressly incorporated herein by reference. Examples of SNRIs include, but are not limited to, mirtazapine, and venlafaxine and pharmaceutically acceptable salts thereof.

The term “antidepressant” as used herein includes compounds that when administered systemically in a mammal, act as corticotropin-releasing factor antagonists (“CRF antagonists”) or that when tested according to standard in vivo or in vitro assays, display receptor-binding properties or other mechanistic properties associated with CRF antagonists. One of skill in the art can readily identify CRF antagonists by in vivo and in vitro assays. For example, CRF antagonists can be identified by adapting the in vitro test method described in U.S. Pat. No. 6,218,391 (issued Apr. 17, 2001), hereby expressly incorporated herein by reference.

Examples of CRF antagonists include, but are not limited to, those described in U.S. Pat. Nos. 6,191,131 (issued Feb. 20, 2001); 6,174,192 (issued Jan. 16, 2001); 6,133,282 (issued Oct. 17,2000); PCT Patent Application Publication Nos. WO 94/13643, WO 94/13644, WO 94/13661, WO 94/13676 and WO 94/13677, and pharmaceutically acceptable salts thereof, all of which patents and publications are hereby expressly incorporated herein by reference.

The term “antidepressant” as used herein includes compounds that when administered systemically in a mammal, act as a-adrenoreceptor antagonists or that when tested according to standard in vivo or in vitro assays, act as α-adrenoreceptor antagonists. One of skill in the art can readily identify a-adrenoreceptor antagonists by in vivo and in vitro assays. For example, a-adrenoreceptor antagonists can be identified by adapting the in vitro test method described in U.S. Pat. No. 6,150,389 (issued Nov. 21, 2000), hereby expressly incorporated herein by reference.

Examples of a-adrenoreceptor antagonists include, but are not limited to, phentolamine and those described in U.S. Pat. No. 6,150,389 and pharmaceutically acceptable salts thereof.

The term “antidepressant” as used herein includes compounds that when administered systemically in a mammal, act as NK1-receptor antagonists (Neurokinin 1 substance P receptor antagonists) or that when tested according to standard in vivo or in vitro assays, act as NK1-receptor antagonists. One of skill in the art can readily identify NK1-receptor antagonists by in vivo and in vitro assays. For example, NK1-receptor antagonists can be identified by adapting the NK1-receptor-binding assay described in U.S. Pat. No. 6,117,855 (issued Sep. 12, 2000), hereby expressly incorporated herein by reference.

Examples of NK1-receptor antagonists include, but are not limited to, those described in PCT Patent Application Publication Nos. WO 95/16679, WO 95/18124, WO 95/23798, and European Patent Specification No. 0 577 394 and pharmaceutically acceptable salts thereof, all of which publications and patent are hereby expressly incorporated herein by reference.

The term “antidepressant” as used herein includes compounds that when administered systemically in a mammal, act as 5-HT_{1A}-receptor agonist, antagonists, and partial agonists (“5-HT_{1A} agents”) or that when tested according to standard in vivo or in vitro assays, act as 5-HT_{1A}-receptor agonist, antagonists, and partial agonists. One of skill in the art can readily identify 5-HT_{1A} agents by in vivo and in vitro assays. For example, 5-HT_{1A} agents can be identified by adapting the 5-HT_{1A} receptor binding assays described in U.S. Pat. No. 6,255,302 (issued Jul. 3, 2001) or 6,239,194 (issued May 29, 2001), which patents are hereby expressly incorporated herein by reference.

Examples of 5-HT_{1A} agents include, but are not limited to, buspirone, flesinoxan, gepirone, and ipsapirone, and pharmaceutically acceptable salts thereof and those disclosed in U.S. Pat. Nos. 6,255,302; 6,245,781 (issued Jun. 12, 2001); and 6,242,448 (issued Jun. 5, 2001). An example of a compound with 5-HT_{1A} receptor antagonist/partial agonist activity is pindolol.

The term “antidepressants” also includes a typical antidepressants. Examples of atypical antidepressants include, but are not limited to bupropion, dimethazan, fencamine, fenpentadiol, levophacetoperance, metralindone, mianserin, cotinine, rolicyprine, rolipram, nefopam, lithium, trazodone, viloxazine, and sibutramine and pharmaceutically acceptable salts thereof.

The term “antidepressants” also includes a wide variety of other drugs that are thought to have antidepressant activity including, but not limited to, nomifensine, oxitriptan, oxypertine, thiazesim, adrafinil, benactyzine, butacetin, dioxadrol, febarbamate, hematoporphyrin, minaprine, piberaline, pyrisuccideanol, roxindole, rubidium chloride, sulpride, thozalinone, tofenacin, l-tryptophan, alaproclate, amitriptyline-chlordiazepoxide combination, atipamezole, azamianserin, bazinaprine, befuraline, binodaline, bipenamol, cericlamine, cianopramine, cimoxatone, clemeprol, clovoxamine, dazepinil, deanol, enefexine, estazolam, fezolamine, fluotracen, idazoxan, levoprotiline, litoxetine, montirelin, nebracetam, norfluoxetine, orotirelin, oxaflozane, pinazepam, pirlindone, setiptiline, sulbutiamine, sulpiride, teniloxazine, thymoliberin, tiflucarbine, tofisopam, tomoxetine, veralipride, viqualine, zimelidine and zometapine, and pharmaceutically acceptable salts thereof, and St. John's wort herb or hypericum perforatum, or extracts thereof.

4. NMDA-Receptor Antagonists as Local Anesthetics

Compounds that act as NMDA-receptor antagonists and pharmaceutically acceptable salts thereof are known to have local-anesthetic properties when administered intradermally and topically. The NMDA receptor is a cell-surface protein complex, widely distributed in the mammalian central nervous system that belongs to the class of ionotropic-glutamate receptors. It is involved in excitatory-synaptic transmission and the regulation of neuronal growth. The structure comprises a ligand-gated/voltage-sensitive ion channel. The NMDA receptor is highly complex and is believed to contain at least five distinct binding (activation) sites: a glycine-binding site, a glutamate-binding site (NMDA-binding site); a PCP-binding site, a polyamine-binding site, and a zinc-binding site. In general, a receptor antagonist is a molecule that blocks or reduces the ability of an agonist to activate the receptor. As used herein, an “NMDA-receptor antagonist” means any compound or composition, known or to be discovered, that when contacted with an NMDA receptor in vivo or in vitro, inhibits the flow of ions through the NMDA-receptor ion channel.

NMDA-receptor antagonist suitable for use in the invention can be identified by testing NMDA-receptor antagonist for local-anesthetic and peripheral antinociceptive properties according to standard pain models. See e.g., J. Sawynok et al., 82 PAIN 149 (1999); J. Sawynok et al., 80 PAIN 45 (1999).

Preferably, the NMDA-receptor antagonist is a non-competitive NMDA-receptor antagonists, more preferably, ketamine, even more preferably, ketamine hydrochloride.

As used herein the meaning of the phrase “NMDA-receptor antagonist” encompasses any compound or composition that antagonizes the NMDA receptor by binding at the glycine site. For a review on glycine-site NMDA-receptor antagonists, see LEESON, P. D., GLYCINE SITE N-METHYL-D-ASPARTATE RECEPTOR ANTAGONISTS, Chapter 13 in DRUG DESIGN FOR NEUROSCIENCE, (Kozikowski, A. P. ed. 338-381, 1993). Glycine-site NMDA-receptor antagonists can be identified by standard in vitro and in vivo assays. See, for example, the assays described in U.S. Pat. No. 6,251,903 (issued Jun. 26, 2001); U.S. Pat. No. 6,191,165 (issued Feb. 20,2001; Grimwood et al. 4 MOLECULAR PHARMACOLOGY 923 (1992); Yoneda et al. 62 J. NEUROCHEM. 102 (1994); and Mayer et al. J. NEUROPHYSIOL. 645 (1988), all of which citations are hereby expressly incorporated herein by reference.

Glycine-site NMDA-receptor antagonists include, but are not limited to, glycinamide, threonine, D-serine, felbamate, 5,7-dichlorokynurenic acid, and 3-amino-1-hydroxy-2-pyrrolidone (HA-966), diethylenetriamine, 1,10-diaminodecane, 1,12-diaminododecane, and ifenprodil and those described in U.S. Pat. Nos. 6,251,903; 5,914,403 (issued Jun. 22, 199); 5,863,916 (issued Jan. 26, 1999); 5,783,700 (issued Jul. 21, 1998); and 5,708,168 (issued Jan. 13, 1998), all of which patents are hereby expressly incorporated herein by reference.

As used herein the meaning of the phrase “NMDA-receptor antagonist” encompasses any compound or composition that antagonizes the NMDA receptor by binding at the glutamate site also referred to herein as “competitive NMDA-receptor antagonists”; see, for example, Olney & Farber, 13 NEUROPSYCHOPHARMACOLOGY 335 (1995).

Competitive NMDA antagonists include, but are not limited to, 3-((−)-2-carboxypiperazin-4-ylpropyl-1-phosphate (CPP); 3-(2-carboxypiperzin-4-yl)-prpenyl-1-phosphonate (CPP-ene); 1-(cis-2-carboxypiperidine-4-yl)methyl-1-phosphonic acid (CGS 19755), D-2-Amino-5-phosphonopentanoic acid (AP5); 2-amino-phosphonoheptanoate (AP7); D, L-(E)-2-amino-4-methyl-5-phosphono-3-pentenoic acid carboxyethyl ester (CGP39551); 2-amino-4-methyl-5-phosphono-pent-3-enoic acid (CGP 40116); (4-phosphono-but-2-enylamino)-acetic acid (PD 132477); 2-amino-4-oxo-5-phosphono-pentanoic acid (MDL 100,453); 3-((phosphonylmethyl)-sulfinyl)-D, L-alanine; amino-(4-phosphonomethyl-phenyl)-acetic acid (PD 129635); 2-amino-3-(5-chloro-1-phosphonomethyl-1H-benzoimidazol-2-yl)-propionic acid; 2-amino-3-(3-phosphonomethyl-quinoxalin-2-yl)-propionic acid; 2-amino-3-(5-phosphonomethyl-biphenyl-3-yl)-propionic acid (SDZ EAB 515); 2-amino-3-[2-(2-phosphono-ethyl)-cyclohexyl]-propionic acid (NPC 17742); 4-(3-phosphono-propyl)-piperazine-2-carboxylic acid (D-CPP); 4-(3-phosphono-allyl)-piperazine-2-carboxylic acid (D-CPP-ene); 4-phosphonomethyl-piperidine-2-carboxylic acid (CGS 19755); 3-(2-phosphono-acetyl)-piperidine-2-carboxylic acid (MDL 100,925); 5-phosphono-1,2,3,4-tetrahydro-isoquinoline-3-carboxylic acid (SC 48981); 5-(2-phosphono-ethyl)-1,2,3,4-tetrahydro-isoquinoline-3-carboxylic acid (PD 145950); 6-phosphonomethyl-decahydro-isoquinoline-3-carboxylic acid (LY 274614); 4-(1H-tetrazol-5-ylmethyl)-piperidine-2-carboxylic acid (LY 233053 and 235723); 6-(1H-Tetrazol-5-ylmethyl)-decahydro-isoquinoline-3-carboxylic acid (LY 233536). References that disclose other competitive NMDA-receptor antagonists as well as assays for identifying competitive NMDA-receptor antagonists include Jia-He Li, et al., 38 J. MED. CHEM. 1955 (1995); Steinberg et al., 133 NEUROSCI. LETT. 225 (1991); Meldrum et al., 11 TRENDS PHARMACOL. SCI., 379 (1990); Willetts et al., 11 TRENDS PHARMACOL. SCI. 423 (1990); Faden et al, 13 TRENDS PHARMACOL. SCI. 29 (1992); Rogawski 14 TRENDS PHARMACOL. SCI. 325 (1993); Albers et al, 15 CLINICAL NEUROPHARM. 509 (1992); Wolfe et al., 13 AM. J EMERG. MED., 174 (1995); and Bigge, 45 BIOCHEM. PHARMACOL. 1547 (1993), all of which citations are hereby expressly incorporated herein by reference.

As used herein the meaning of the phrase “NMDA-receptor antagonist” encompasses any compound or composition that antagonizes the NMDA receptor by binding at the PCP (phencyclidine) site, referred to herein as “non-competitive NMDA-receptor antagonists”; see, for example, Bigge 45 BIOCHEM. PHARMACOL. 1547 (1993).

Non-competitive NMDA-receptor antagonists can be identified using routine assays, for example, those described in U.S. Pat. Nos. 6,251,948 (issued Jun. 26, 2001); 5,985,586 (issued Nov. 16, 1999), and 6,025,369 (issued Feb. 15, 2000); Jacobson et al., 110 J. PHARMACOL. EXP. THER. 243 (1987); and Thurkauf et al., 31 J. MED. CHEM. 2257 (1988), all of which citations are hereby expressly incorporated herein by reference.

Examples of non-competitive NMDA-receptor antagonists that bind at the PCP site include, but are not limited to, ketamine, phencyclidine, dextromethorphan, dextrorphan, dexoxadrol, dizocilpine (MK-801), remacemide, thienylcyclohexylpiperidine (TCP), N-allylnormetazocine (SKF 10,047), cyclazocine, etoxadrol, (1,2,3,4,9,9a-hexahydro-fluoren-4a-yl)-methyl-amine (PD 137889); (1,3,4,9,10,10a-hexahydro-2H-phenanthren-4a-yl)-methyl-amine (PD 138289); PD 138558, tiletamine, kynurenic acid, 7-chloro-kynurenic acid, and memantine; and quinoxalinediones, such as 6-cyano-7-nitroquinoxaline-2,3-dione (CNQX) and 6,7-dinitro-quinoxaline-2,3-dione (DNQX) and pharmaceutically acceptable salts thereof.

As used herein the meaning of “NMDA-receptor antagonist” encompasses compounds that block the NMDA receptor at the polyamine binding site, the zinc-binding site, and other NMDA-receptor antagonists that are either not classified herein according to a particular binding site or that block the NMDA receptor by another mechanism. Examples of NMDA-receptor antagonists that bind at the polyamine site include, but are not limited to, spermine, spermidine, putrescine, and arcaine. Examples of assays useful to identify NMDA-receptor antagonists that act at the zinc or polyamine binding site are disclosed in U.S. Pat. No. 5,834,465 (issued Nov. 10, 1998), hereby expressly incorporated by reference herein.

Other NMDA-receptor antagonists include, but are not limited to, amantadine, eliprodil, iamotrigine, riluzole, aptiganel, flupirtine, celfotel, levemopamil, 1-(4-hydroxyphenyl)-2-(4-phenylsulfanyl-piperidin-1-yl)-propan-1-one; 2-[4-(4-fluoro-benzoyl)-piperidin-1-yl]-1-naphthalen-2-yl-ethanone (E 2001); 3-(1,1-dimethyl-heptyl)-9-hydroxymethyl-6,6-dimethyl-6a,7,8,10a-tetrahydro-6H-benzo[c]chromen-1-ol (HU-211); 1-{4-[1-(4-chloro-phenyl)-1-methyl-ethyl]-2-methoxy-phenyl}-1H-[1,2,4]triazole-3-carboxylic acid amide (CGP 31358); acetic acid 10-hydroxy-7,9,7′,9′-tetramethoxy-3,3′-dimethyl-3,4,3′,4′-tetrahydro-1H,1H-[5,5′]bi[benzo[g]isochromenyl]-4-yl ester (ES 242-1); 14-hydroxy-11-isopropyl-10-methyl-5-octyl-10,13-diaza-tricyclo[6.6.1.04,15]pentadeca-1,4,6,8(15)-tetraen-12-one; and 4,5-dioxo-4,5-dihydro-1H-benzo[g]indole-2,7,9-tricarboxylic acid (PQQ) and pharmaceutically acceptable salts thereof.

5. Other Local-Anesthetic Agents

Other agents with local-anesthetic properties include analgesics, such as non-steroidal anti-inflammatories (“NSAIDs”), see, for example, TRANSDERMAL AND TOPICAL DRUG DELIVERY SYSTEMS 87-93 (Tapash K. Ghosh et al. eds., 1997). Examples of non-narcotic analgesics with local-anesthetic properties include, but are not limited to, acetylsalicylic acid, ketoprofen, piroxicam, diclofenac, indomethacin, and ketorolac.

In yet another embodiment of the current invention, agents may be included in the patches of the invention to prolong the local-anesthetic effect, such as, a glucocorticosteroid (see, for example, U.S. Pat. No. 5,922,340, incorporated herein by reference) or a vasoconstrictor, such as a catecolamine.

6. Mixtures of Local-Anesthetic Agents

Combinations of one or more local anesthetics can also be used in patches of the invention. But one example is a combination of an NMDA receptor antagonist and an antidepressant, preferably, a non-competitive NMDA receptor antagonist, such as ketamine or a pharmaceutically acceptable salt thereof and a tricyclic antidepressant, such as amitriptyline or a pharmaceutically acceptable salt thereof. Another example of a mixture of local anesthetics useful in patches of the invention is a combination of an opioid and a sodium-channel blocker, such as a mixture of morphine or a pharmaceutically acceptable salt thereof and lidocaine or a pharmaceutically acceptable salt thereof.

B. The Breathable Backing Layer

Patches of the invention comprise a backing layer that is a breathable (i.e., air and water vapor permeable), electron-beam stable, γ-radiation stable, and that adheres to the hydrogel-local-anesthetic mixtures described herein. Breathable backings allow the skin-application site to breath (exchange of oxygen and carbon dioxide) and allows water-vapor transmission from the skin surface. Such characteristics are essential for treating the pain associated with non-intact skin indications, such as open and closed wounds and burns, to prevent infection. Preferably, backings used in patches of the invention have a thickness within the range of from about 15 μm to about 125 μm.

Permeability of backings for use in patches of the invention can be expressed as the moisture-vapor-transmission rate (“MVTR”), which represents the rate that moisture permeates through a barrier expressed in units of grams/meter²/day (“g/m²/d”). Preferably, the breathable backing displays a MVTR value from about 500 to about 5000 g/m²/d measured according to ASTM F1249 (MOCON), more preferably, the breathable backing displays a MVTR value of about 1,000 g/m²/d.

Suitable backing materials are readily identified by one of skill in the art by measuring the potential backing's MVTR value, evaluating its compatibility with and adhesion to the hydrogel-local anesthetic mixture, and by testing the backing's stability to γ-radiation sterilization. Examples of suitable backing materials include, but are not limited to, copolyesters, polyether/polyamide copolymers, polyurethanes, and polyethylene derivatives. Examples of suitable polyether/amide copolymers include, but are not limited to, PEBAX®, commercially available from Atochem Inc. of Glen Rock, N.J. Examples of suitable polyurethanes include, but are not limited to, ESTANE, commercially available from The B. F. Goodrich Company of Cleveland, Ohio. Examples of suitable polyethylene derivatives include, but are not limited to, SKYCARE AND SCYAIR films, commercially available from Skymark Performance Films Ltd., North Lincolnshire, UK.

In a preferred embodiment, the backings of the invention are medical grade copolyester film. A copolyester elastomer is a block copolymer consisting of aliphatic diols, aromatic diacids, and polyalkylene ether-diols. 19 KIRK-OTHMER ENCYCLOPEDIA OF CHEMICAL TECHNOLOGY 632 (4th ed. 1996). Preferably, the copolyester is HYTREL®. The HYTRELs are a series of polyester/polyether copolymers comprising a hard (crystalline) segment of polybutylene terephthalate and a soft (amorphous) segment of long-chain polyether glycols. In general, the ratio of soft to hard segments determines the elasticity of the copolyester. HYTRELs are commercially available from DuPont, Clopay Corporation, Cincinnati, Ohio.

Copolyesters, such as HYTREL, are generally obtained as a polymer pellets, which are then processed into films using well-known film extrusion processes. The extruded films are then ready for use in patches of the invention. A preferred extruded HYTREL film is commercially available from Mylan Technologies, Inc. (St. Albans, Vt.) under the name MEDIFILM®325. This particular backing has a thickness of about 0.05 mm and an MVTR of 1044 g/m²/day as measured by ASTM F1249.

C. Hydrogels

Any hydrogel that is y-radiation sterilizable and can intradermally deliver a local anesthetic is suitable for use in patches of the invention. Preferably, the hydrogel is compatible with and promotes healing of wounds. The hydrogel should be of sufficient tackiness to adhere the patch to the application site but also be removable without irritation or wound damage. Preferably, the hydrogel has a water content of from about 60% to about 90% by weight, more preferably, about 80% and a tackiness wherein the tack-rolling-ball distances are of at least about 5 mm as measured by the rolling-ball test described in Section 5C1a below.

Preferably, the hydrogel is polyvinylpyrrolidone (“PVP”) of an average molecular weight of about 500,000 Daltons to about 2,000,000 Daltons, more preferably, about 900,000 Daltons to about 1,500,000 Daltons that has been electron-beam cross-linked. In a preferred embodiment, the hydrogel comprises cross-linked polyvinylpyrrolidone, a preservative, water, and a local anesthetic. Other excipients and pharmaceuticals may be incorporated in the hydrogel.

1. Physical Characteristics of Hydrogels

a. Tackiness

Tackiness of hydrogels can be measured according to the tack rolling ball (“TRB”) test detailed in The American Society for Testing Materials (ASTM), Designation: D 3121-94 (Reapproved 1999) “Standard Test for Tack of Pressure-Sensitive Adhesives by Rolling Ball”, hereby incorporated herein by reference. Preferably hydrogels employed in the patches of the invention have TRB ranging from about 5 mm to about 20 mm, preferably, about 7 mm to about 15 mm. Such TRB values indicate hydrogels of sufficient tack to adhere to skin but not enough to irritate the application site upon removal.

Suitable apparatus for performing the test is available from the Pressure Sensitive Tape Council, The Breeden Co., Deerfield, Ill. The test is run at 72° F.±5° F. and 50%±10% relative humidity. The hydrogel sample (about 2″ wide and about 15″ long) is placed on a clean metal or glass plate, adhesive side up, in line with a TBR inclined trough equipped with a release lever. Clean, dry tongs are used to place a 11.1 mm steel ball on the TBR trough, which is then released. The distance from the point where the ball initially contacts the adhesive to where the ball stops is measured (i.e., the TBR value). The test is repeated at least five times with a clean ball and a fresh hydrogel strip and the average TBR value is recorded. Pertinent additional comments based on visual inspection such as noticeable residue on ball, lift of adhesive from substrate, etc., are recorded.

b. Commercial Hydrogel Sources

Hydrogels suitable for use in patches of the invention are commercially available. For example, suitable hydrogels can be purchased from Hydrogel Design Systems, Langehome, Pa. or Tyco, Inc., Chicopee, Mass.

D. Manufacture of Patches of the Invention

Exemplary procedures for preparing of polyvinylpyrrolidone-hydrogels for use in patches of the invention are described in WO 93/10163 (published May 27, 1993) page 12, line 24 through page 13, line 3; U.S. Pat. No. 4,989,607, column 13, lines 10-25; EP 0 107 376 (published Feb. 5, 1984) page 19, lines 10-30; D. Darwis 42 RADIAT. PHYS. CHEM. 907 (1993); and Olgun Guven & Murat Sen 32 POLYMER 2491 (1991), all of which citations are hereby expressly incorporated herein by reference.

In general, patches of the invention can be prepared as follows. First, a “pre-hydrogel mixture” is prepared comprising a homogeneous mixture of:

(a) about 5% to about 35% by weight, preferably, about 10% to about 30%, more preferably, about 15% to about 20% by weight of USP polyvinylpyrrolidone having an average molecular weight ranging from about 900,000 to about 1,500,000;

(b) about 0.5% to about 20% by weight of a local anesthetic, preferably about 2% to about 10% of a local anesthetic; and

(c) the remainder water.

Preferably, the mixture further comprises a preservative in about 0.1% to about 2% by weight of the hydrogel portion of the patch.

In a suitable vessel—for example, a stainless-steel mixing tank—the water and local anesthetic are blended and the PH adjusted to about 6.3. The USP polyvinylpyrrolidone and preservative are then added and the mixture blended for about 16 hours to about 24 hours. If the resulting mixture is foamy, it can stand for about 5 to 15 days to clarify and allow the foam to settle. Deaeration can be accelerated by vacuum.

The pre-hydrogel mixture as prepared above is then coated, using a slot die, onto a suitable release liner (for example a polyethylene terphthalate sheet 0.003″ treated with silicon, commercially available, for example, from Rayven, Inc., Willow Grove, Pa.) at a thickness ranging from about 0.015″ to about 0.06″, preferably, about 0.025″ to about 0.035″, more preferably, about 0.033″ to form a pre-hydrogel film layer. The pre-hydrogel film layer is then covered with a breathable backing sheet (e.g., an extruded polyester/polyether copolymer film), forming a sandwich (“pre-hydrogel substrate”). The pre-hydrogel substrate is then treated with high-energy radiation to cross link the polyvinylpyrrolidone, thereby forming a hydrogel. The high-energy radiation can be alpha particles, beta particles, gamma rays, X-rays, an electron beam, or high-energy ultraviolet radiation. In a preferred embodiment, an electron-beam is used. The electron beam should be of sufficient energy to completely penetrate the mixture so that the mixture receives a radiation dose effective to cross link the entire cross section of the sample. The radiation dose will vary depending upon the molecular weight of the polyvinylpyrrolidone, its concentration, the thickness, and the presence and identity of other components or additives. The radiation dose can be expressed as that needed to achieve particular hydrogel physical characteristics. For example, a sufficient amount of radiation can be used to prepare a hydrogel of the desired tackiness, cross linking, and absorptive capacity, which parameters can be measured as described above. Proper dose/energy/thickness relationships are readily available to those skilled in the art of radiation processing, for example see WO 93/10163 (published May 27, 1993); U.S. Pat. No. 4,699,146 (issued Aug. 1, 2000). Multiple doses and times of exposures to the electron beam may be used and selection of such parameters is well known in the art. For example, the electron-beam can be operated at a current of about 5 mA to about 30 mA and at a voltage of about 1 MeV and the pre-hydrogel substrate passed under the electron beam at a rate of about 5 to about 25 feet per minute. In general, the radiation dose received by the pre-hydrogel substrate ranges from about 0.5 Mrads to about 4 Mrads, more preferably, about 0.5 Mrads to about 2 Mrads.

The electron beam can be produced by an electron-beam accelerator [commercially available, for example, Radiation Dynamics, Inc.]. For example, a suitable procedure is described in U.S. Pat. No. 4,699,146 (issued Oct. 13, 1987), hereby expressly incorporated herein by reference.

The patch is cut to the desired size and shape using a rotary-die press or clicker press packaged and sterilized.

E. Sterile Packaging

The patches of the invention can be packaged in a sterile environment according to well-known methods. See e.g., 2 REMINGTON: THE SCIENCE AND PRACTICE OF PHARMACY 1463-1494 (Alfonso R. Gennaro ed., 19th ed. 1995), incorporated herein by reference. Preferably, the patches of the invention are sealed in water-vapor impermeable, single-use packages and sterilized with 15-40 kGray y-radiation. Suitable packaging materials include pre-manufactured laminates sealed on three sides, comprising: polyester/aluminum/heat-sealable polyester; paper/aluminum/heat-sealable polyester; polyester/aluminum/polyethylene; or paper/aluminum/polyethylene. Preferably, the thickness of the aluminum layer is from about 6 to about 10 microns. Such laminates are commercially available, for example, from Curwood Industries, WI or Genesis Packaging, CA.

F. Other Components of Patches of the Invention

The patches of the invention can further comprise one or more additional ingredients, such as one or more preservatives, stabilizers, adsorptive agents, wound-healing agents, electrolytes or tonicity agents, viscosity-enhancing agents, medicinal agents, bioadhesive polymers, penetration enhancers, or humectants. One skilled in the art will readily be able to choose such additional excipients based on the physical and chemical properties desired in the patch. Of course, a single excipient may have multiple functions and properties.

1. Preservatives

The patches of the invention can comprise a preservative in the hydrogel layer to retard the growth of bacteria, preferably, in an amount of about 0.1% to about 2% by weight of the hydrogel portion of the patch. In one embodiment, a preservative can be added to the pre-hydrogel mixture during patch manufacture. Preferably, the preservative is stable to electron-beam and y-radiation. Examples of preservatives include, but are not limited to, DOWICIL-2000® (active ingredient: cis 1-(3-chloroallyl)-3,5,7-triaza-1-azonia-adamantane chloride; sold by Dow Chemical Co. Midland, Mich.), methyl paraben, ethyl paraben, propyl paraben, butyl paraben, paraben salts, GLYDANT® (1,3-dimethylol-5,5-dimethyl hydantoin, sold by Lonza Co., Basel, Switzerland), GERMALL PLUS® (99% Germall II (Diazolidinyl Urea) and 1% iodopropynyl butylcarbamate; sold by International Speciality Products, Wayne, N.J.), or combinations thereof. Preferably, the preservative is PHENONIP® (Clariant Corporation, Mount Holly, N.C.), which is a blend of paraben esters in phenoxyethanol.

2. Stabilizers

Stabilizers can be included in patches of the invention to enhance chemical stability. When a stabilizer is included, preferably, it is present in the hydrogel layer. In one embodiment, a stabilizer can be added to the pre-hydrogel mixture during patch manufacture. Examples of stabilizers include, but are not limited to, amino acids; antioxidants, such as ascorbic acid, sodium bisulfite, sodium metabisulfite, thiourea, butylated hydroxytoluene, and tocopherols; chelating agents, such as EDTA; and buffers, such as malic acid, potassium citrate, and sodium phosphate.

3. Adsorptive Agents

Adsorptive agents can be included in patches of the invention to facilitate wound healing by absorbing wound discharge. When an adsorptive agent is included, preferably, it is present in the hydrogel layer. In one embodiment, an adsorptive agent can be added to the pre-hydrogel mixture during patch manufacture. Examples of adsorptive agents include, but are not limited to, cellulose derivatives, bentonite, cellulose, silicon dioxide, kaolin, and magnesium aluminum silicate.

4. Wound-Healing Agents

Wound healing involves five phases: (1) injury, (2) coagulation, (3) inflammation, (4) tissue formation, and (5) tissue remodeling. Upon injury, injured cells release cytokines, which initiate events that lead to wound healing. Coagulation occurs immediately after injury via platelet agglutination at the injury site. A fibin clot forms via the activation of the coagulation cascade. Thrombin induces platelet degranulation, leading to the release of growth factors and adhesive glycoproteins. The fibrin clot acts as a matrix for colonization by inflammatory cells. Inflammation occurs one to five days after injury. Migrating inflammatory cells accumulate in the healing wound. Macrophages are the most important inflammatory cell in wound healing. They provide wound decontamination. Macrophage-derived cytokines are essential for the initiation and propagation of new tissue formation at the wound site. Macrophages facilitate the transition from the inflammatory phase to the tissue-repair phase. Tissue formation occurs between days three to twelve. Re-epithelialization begins at the wound edges reestablishing the integrity of the dermis and epidermis. Wound contraction reaches its peak at about five to fifteen days after injury. Tissue remodeling can continue for a year or more depending on the wound's severity in an attempt to return the wounded area to its normal tissue structure.

Wound healing agents can be included in patches of the invention to promote wound healing and to mitigate scarring. The phrases “promote wound healing,” means either the induction of the formation of granulation tissue of wound contraction and/or the induction of epithelialization (i.e., the generation of new cells in the epithelium).

Wound healing agents include growth factors, such as PDGF, TGF-β, EGF, TGF-α, KGF, IL-1, FGF, TNF-α, IGF-1, IFNs, which are effective at various stages of the wound-healing process; agents that enhance epidermal resurfacing, such as benzoyl peroxide, allantoin, zinc oxide, and cod liver oil; corticosteroids, such as 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortisol, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, flucortin butyl, fluocortolone, fluorometholone, fluperolone acetate, fluprednidene acetate, fludrocortisone, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide; colchicine; dapsone; antimalarials, such as acedapsone, amodiaquin, arteether, chirata, chloroquine, cinchone, cinchonidine, cycloguanil, plasmocid, quinidine, quinine, quinocide, and quinoline; retinoids, such as vitamin A; vitamin E; angiotensin II and fragments thereof as described in U.S. Pat. No. 6,096,709 (hereby incorporated herein by reference); the peptides described in U.S. Pat. No. 6,248,716, column 2, lines 35-61 (hereby incorporated herein by reference); the compounds described in U.S. Pat. No. 6,194,578 column 1, line 43 through column 5, line 63 (hereby incorporated herein by reference); adenosine receptor agonists, for example, adenosine, 2-phenylaminoadenosine, 2-para-2carboxyethylphenylamino-5′-ethylcarboxamidoadenosine, 5′N-cyclopropyladenosine, 5′-N-methylcarboxamidoadenosine and PD-125944; agonists of adrenergic β-3 receptors, such as those disclosed in U.S. Pat. No. 6,235,793, column 1 line 50 though column 4 line 59 (hereby incorporated herein by reference); compounds having oxytocin activity, such as those disclosed in U.S. Pat. No. 6,262,021 column 5, lines 1-63 (hereby incorporated herein by reference); gibberellins, such as those disclosed in U.S. Pat. No. 6,121,317, column 2, lines 1-37 (hereby incorporated herein by reference); chrysalin (Abbot Laboratories); thymosin 4; and becaplermin.

5. Electrolytes and Tonicity Agents

Electrolytes and tonicity agents can be included in the hydrogel layer of patches of the invention. In one embodiment, an electrolyte or tonicity agent can be added to the pre-hydrogel mixture during patch manufacture. Suitable electrolytes include most cations, e.g., ammonium, sodium, potassium, lithium, magnesium, calcium, etc., and both simple and complex anions, e.g., chloride, sulfate, carbonates, nitrates, and anions of organic acids, e.g., acetic, citric, adipic, tartaric, lactic, propionic, glutaric and maleic acids. Examples of tonicity agents include, but are not limited to, amino acids, fdextrose, glycerol, potassium chloride, and sodium chloride.

6. Viscosity-Enhancing Agents

The hydrogel layer of patches of the invention can include viscosity enhancing agents, such as hydrophilic polymers. When used, preferably, the viscosity-enhancing agent is added to the pre-hydrogel mixture during patch manufacture. The introduction of a hydrophilic polymer having a weight average molecular weight in excess of about 100 kilodaltons, in a few percent, can enhance the viscosity of the hydrogel to modify its coatability and extrudability. Typically, when included, the viscosity enhancing agent is added to the pre-hydrogel mixture in about 1% to about 2% by weight of the hydrogel portion of the patch. In general, viscosity enhancing polymers should have an average molecular weight in excess of about 100,000 Daltons. Examples of viscosity-enhancing agents include, but are not limited to, polyacrylamide, poly(vinyl alcohol), poly(ethylene imine), polyacrylamide sulfonic acid or their salts, polyacrylonitrile, starch, agar, dextran, dextrins and derivatives, starch derivatives, carrageenan, xanthan, and guar.

7. Medicinal Agents

The patches of the invention can include medicinal agents or their pharmaceutically acceptable salts. Medicinal agents are compounds that upon transdermal or intradermal adsorption have a pharmaceutical effect. When used, preferably, the medicinal agent is added to the pre-hydrogel mixture during patch manufacture. One of skill in the art can readily choose a medical agent to incorporate into the patches of the invention and its appropriate concentration depending on the indication and desired effect. Examples of medicinal agents include, but not limited to, non-steroidal anti-inflammatories, such as acetaminophen, aspirin, ibuprofen, diclofenac, nabumetone, misoprostol, oxaprozin, piroxicam, and etodolac; antifingals such as ciclopirox, chloroxylenol, triacetin, sulconazole, nystatin, undecylenic acid, tolnaftate, miconizole, clotrimazole, oxiconazole, griseofulvin, econazole, ketoconozole, and amphotericin B; antibiotics, such as neomycin, polymyxin B, gentamicin, bacitracin, mupirocin, silver sulfadiazine, erthromycin, and clindamycin; antiseptics, such as iodine, povidine-iodine, benzalkonium chloride, benzoic acid, chlorhexidine, nitrofurazone, benzoyl peroxide, hydrogen peroxide, hexachlorophene, phenol, resorcinol, and cetylpyridinium chloride; and anti-inflammatories, such as hydrocortisone, prednisone, triamcilolone, betamethasone, dexamethasone.

8. Bioadhesive Polymers

The patches of the invention can include one or more bioadhesive polymers. Bioadhesive polymers hydrate the skin and can also function as thickening agents. When used, preferably, the bioadhesive polymer is added to the pre-hydrogel mixture during patch manufacture. Examples of bioadhesive polymers include, but are not limited to, pectin, alginic acid, chitosan, hyaluronic acid, polysorbates, such as polysorbate-20, -21, -40, -60, -61, -65, -80, -81, -85; poly(ethyleneglycol), such as PEG-7, -14, -16, -18, -55, -90, -100, -135, -180, -4, -240, -6, -8, -9, -10, -12, -20, or -32; oligosaccharides and polysaccharides, such as gellan, carrageenan, xanthan gum, gum Arabic, and dextran; cellulose esters and cellulose ethers; modified cellulose polymers, such as carboxymethylcellulose, hydroxyethylcellulose, hydroxypropyl methylcellulose, hydroxyethyl ethylcellulose; polyether polymers and oligomers, such as polyoxyethylene; condensation products of poly(ethyleneoxide) with various functionalized hydrocarbons (e.g. aliphatic chains of about 12 to 20 carbon atoms), for example, the condensation product of poly(ethylene oxide) with fatty acids, fatty alcohols, fatty amides, or polyhydric alcohols; polyether compounds, such as poly(methyl vinyl ether) and polyoxypropylene; polyether compounds, such as block copolymers of ethylene oxide and propylene oxide; pluronic lethicin organogel (see 1 INTERNATIONAL JOURNAL OF PHARMACEUTICAL COMPOUNDING 71 (1997)); poly(vinyl alcohol); polyacrylamide; polyvinylpyrrolidone; polymethacrylic acid; polyacrylic acid or coss-linked polyacrylic acid, such as carbomer, i.e., a homopolymer of acrylic acid cross linked with either an allyl ether of pentaerythritol, an allyl ether of sucrose, or an allyl ether of propylene (e.g., Acrisint® 400, 410, or 430 commercially available from 3V Inc. Weehawkin, N.J.); Orabase® (i.e., a mixture of gelatine, pectin, and sodium carboxymethyl cellulose in a plasticized hydrocarbon gel, commercially available from Hoyt laboratories, Needhm, Mass.); and Carafateg (sulfated sucrose and aluminum hydroxide, commercially available from Marion Laboratories, Inc., Kansas City, Mo.).

9. Penetration Enhancers

In another embodiment, the patches of the invention can further comprise a penetration enhancer. When present in patches of the invention, the penetration enhancer is added to the pre-hydrogel mixture in an amount of from about 0.1% to about 5% by weight, more preferably from about 1% to about 2% by weight.

Penetration enhancers can be included in the patches of the invention to optimize transfer of the local anesthetic through the stratum comeum and into the dermis to provide a local effect. For a discussion of use of penetration enhancers in topical formulations see generally, PERCUTANEOUS PENETRATION ENHANCERS (Eric W. Smith & Howard I. Maibach eds. 1995); Ghosh, T. K. et al. 17 PHARM. TECH. 72 (1993); Ghosh, T. K. et al. 17 PHARM. TECH. 62 (1993); Ghosh, T. K. et al. 17 PHARM. TECH. 68 (1993), all of which citations are hereby incorporated herein by reference. The penetration enhancer should be pharmacologically inert, non-toxic, and non-allergenic, have rapid and reversible onset of action, and be compatible with the patches of the invention.

Examples of penetration enhancers include, but are not limited to, transcutol P, ethyl alcohol, isopropyl alcohol, lauryl alcohol, salicylic acid, octolyphenylpolyethylene glycol, polyethylene glycol 400, propylene glycol, N-decylmethylsulfoxide, DMSO, glycerin, octolyphenylpolyethylene glycol, oleic acid, polyethylene glycol, propylene glycol, N-decylmethylsulfoxide, isopropyl myristate, methyl laurate, glycerol monooleate, propylene glycol monooleate, and N-methyl pyrrolidone.

10. Humectants

Humectants can be included in the hydrogel layer of patches of the invention. When used, preferably, the humectant is added to the pre-hydrogel mixture during patch manufacture. Humectants include, but are not limited to, glycerol, propylene glycol and polyethylene glycol. Additional agents, such as polyfunctional crosslinking promoters may be added to overcome the resistance to crosslinking resulting from the use of humectants. These agents include acrylic or methacrylic monomer derivatives.

G. Indications

The patches of the invention are particularly effective for treating or preventing the pain associated with non-intact skin indications, such as wounds and burns.

The patches of the invention, however, can be used to treat or prevent any indication resulting from noxious stimulation of peripheral nociceptors. The patches and methods of the invention are effective to induce local anesthesia and to treat neuropathic pain. As used herein the term “neuropathic pain” refers to neuropathic-pain syndromes, that is, pain due to lesions or dysfunction in the nervous system. The patches and methods of the invention can be used to treat or prevent pain related to or induced by the following diseases, trauma, or conditions: general neuropathic conditions, such as peripheral neuropathy, phantom pain, reflex-sympathetic dystrophy, causalgia, syringomyelia, and painful scar; specific neuralgias at any location of the body; back pain; diabetic neuropathy; alcoholic neuropathy; metabolic neuropathy; inflammatory neuropathy; chemotherapy-induced neuropathy, herpetic neuralgias; traumatic odontalgia; endodontic odontalgia; thoracic-outlet syndrome; cervical, thoracic, or lumbar radiculopathies with nerve compression; cancer with nerve invasion; traumatic-avulsion injuries; mastectomy, thoracotomy pain; spinal-cord-injury; stroke; abdominal-cutaneous nerve entrapments; tumors of neural tissues; arachnoiditis; stump pain; fibromyalgia; regional sprains or strains; myofascial pain; psoriatic arthropathy; polyarteritis nodosa; osteomyelitis; burns involving nerve damage; AIDS-related pain syndromes; connective tissue disorders, such as systemic lupus erythematosis, systemic sclerosis, polymyositis, and dermatomyositis; and inflammatory conditions, such as acute inflammation (e.g. trauma, surgery and infection) or chronic inflammation (e.g., arthritis and gout).

H. Dosage and Application

Selection of the appropriate dosage of local anesthetic for the application site is an important consideration. The rate of intradermal anesthetic delivery from a patch of the invention is a function of the application site, for example, whether the patch is to applied to intact skin or to a wound or burn. The dosages and dosing frequency will be determined by a trained medical professional.

When a patch is used to relieve the pain from a wound or burn, the dosage of the local anesthetic required to achieve pain relief is determined by the active surface area of the patch in direct contact with the wound. The patch should cover at least the entire wound area. In general, a physician may begin dosing with a low or intermediate strength patch (local anesthetic in an amount of about 2% to about 10% by weight of the hydrogel portion of the patch) and then, depending upon the effectiveness, adjust the dosage up or down by prescribing a patch of higher or lower anesthetic concentration or recommend the use of a different local anesthetic. Fresh patches may be applied multiple times per day, preferably, a fresh patch is applied about every 4 to about every 48 hours. More preferably, the patch is applied daily.

Wounds are likely to be contaminated, thus, require thorough cleansing to remove foreign materials and bacteria. Cleansing should not cause further tissue damage. Irrigation with water, saline solution, or a non-toxic antiseptic solution (e.g., hibitane solution or povidone/iodine).

A sterilely packaged patch of the invention is ready for use, it is removed from its package, the release liner is removed by peeling it from the gel, and it is topically applied to the application site. The patches of the invention should be applied using a sterile technique.

The present invention and its many attendant advantages will be understood from the foregoing description and it will be apparent that various changes in form, construction and arrangement of the parts thereof may be made without departing from the spirit and scope of the invention or sacrificing all of its material advantages, the form hereinbefore described are merely exemplary embodiments thereof.

### VI. EXAMPLES

### Example 1

### Manufacture of a Patches of the Invention

In a stainless-steel mixing tank the water and lidocaine hydrochloride in the amounts specified in Table 1 were blended and the PH was adjusted to 6.3±0.2 with. USP polyvinylpyrrolidone (medical grade; commercially available, for example, PVP K90 from BASF Corporation, Mount Olive, N.J.) and PHENONIP® were then added in the amounts specified in Table 1 and the mixture blended for about 24 hours to give a foamy product about the consistency of honey. The solution was allowed to stand for about 15 days to clarify and allow the foam to settle.

The homogeneous polyvinylpyrrolidone-local anesthetic mixture as prepared above was then coated, using a slot die, at a thickness of, about 0.033″ on a 0.003″ polyethylene terphthalate sheet of treated with silicon, commercially available, for example, from Rayven, Inc., Willow Grove, Pa.). The polyvinylpyrrolidone-local anesthetic mixture was then covered with 0.002″ thick Mylan Medifilm 325 (Mylan Technologies, Inc., St. Albans, Vt.), forming a sandwich. The PVP-lidocaine sandwich was conveyed, at a rate of 20 feet per minute, through an electron-beam generated by a Dynamitron accelerator operated at a voltage of about 1 MeV at the current indicated in Table 1.

TABLE 1

| | wt | wt % | wt % | hydrogel | electron-beam |
|---|---|---|---|---|---|
| Patch | % PVP | lidocaine | PHENONIP ® | thickness | current |
| 1 | 20% | 4% | 0.5% | 0.035″ | 8.1 mA |
| 2 | 20% | 4% | 0.5% | 0.025″ | 8.1 mA |
| 3 | 25% | 4% | 0.5% | 0.035″ | 12 mA |
| 4 | 25% | 4% | 0.5% | 0.025″ | 12 mA |
| 5 | 15% | 4% | 0.5% | 0.035″ | 8.1 mA |
| 6 | 15% | 4% | 0.5% | 0.025″ | 8.1 mA |
| 7 | 20% | 10% | 0.5% | 0.035″ | 16 mA |
| 8 | 20% | 10% | 0.5% | 0.025″ | 16 mA |
| 9 | 25% | 10% | 0.5% | 0.035″ | 12 mA |
| 10 | 25% | 10% | 0.5% | 0.025″ | 12 mA |
| 11 | 15% | 10% | 0.5% | 0.035″ | 16 mA |
| 12 | 15% | 10% | 0.5% | 0.025″ | 16 mA |
| 13 | 20% | 20% | 0.5% | 0.035″ | 20 mA |
| 14 | 20% | 20% | 0.5% | 0.025″ | 20 mA |
| 15 | 25% | 20% | 0.5% | 0.035″ | 16 mA |
| 16 | 25% | 20% | 0.5% | 0.025″ | 16 mA |
| 17 | 15% | 20% | 0.5% | 0.035″ | 20 mA |
| 18 | 15% | 20% | 0.5% | 0.025″ | 20 mA |

The resulting patch was cut to a size of 1.5″×7.125″ using a rotary die press (commercially available, for example, from Mark Andy, Inc., Chesterfield, MOand packaged in a 0.10 mm polyethylene bag. The packaged patch was sterilized with about 20 to 40 kGray of y-irradiation from a cobalt source using well-known methods.

### Example 2

### Treatment of a Burn or Wound with a Patch of the Invention

After selection of appropriate dosage and size, a sterile patch as manufactured in Example 1 is removed from the package by the patient or doctor and the release liner is peeled exposing the hydrogel. The patch is placed over the bum or wound such that the entire wound and about 1 mm to about 5 mm of the surrounding uninjured skin is covered. If desired, an overlap of non-woven polyester having a suitable medical grade adhesive on one side, may be placed over the patch for additional stability. The patch may be removed and replaced as needed.

The present invention is not to be limited in scope by the specific embodiments disclosed in the examples, which are intended as illustrations of a few aspects of the invention, and any embodiments that are functionally equivalent are within the scope of this invention. Indeed, various modifications of the invention in addition to those shown and described herein will become apparent to those skilled in the art and are intended to fall within the scope of the appended claims.

All cited references are hereby incorporated herein in their entireties by reference.

What is claimed is:

1. A patch comprising a breathable backing coated with a polyvinylpyrrolidone-based hydrogel, the hydrogel comprising one or more local anesthetics or a pharmaceutically acceptable salt thereof.

2. The patch of claim 1, wherein the patch is sterile.

3. The patch of claim 1, wherein the breathable backing comprises a polyester/polyether copolymer film.

4. The patch of claim 1, wherein the hydrogel further comprises a preservative.

5. The patch of claim 1, wherein the local anesthetic comprises a sodium-channel blocker, an antidepressant, an NMDA receptor antagonist, or an opioid, or a pharmaceutically acceptable salt thereof or a mixture thereof.

6. The patch of claim 5, wherein the sodium-channel blocker is lidocaine or a pharmaceutically acceptable salt thereof.

7. The patch of claim 5, wherein the antidepressant is a tricyclic antidepressant or a pharmaceutically acceptable salt thereof.

8. The patch of claim 5, wherein the antidepressant is amitriptyline or a pharmaceutically acceptable salt thereof.

9. The patch of claim 5, wherein the NMDA-receptor antagonist is a non-competitive NMDA-receptor antagonist or a pharmaceutically acceptable salt thereof.

10. The patch of claim 5, wherein the NMDA-receptor antagonist is ketamine or a pharmaceutically acceptable salt thereof.

11. The patch of claim 5, wherein the opioid is morphine or a pharmaceutically acceptable salt thereof.

12. A package containing a sterile patch, the patch comprising a breathable backing coated with a polyvinylpyrrolidone-based hydrogel, the hydrogel comprising one or more local anesthetics or a pharmaceutically acceptable salt thereof.

13. The package of claim 12, wherein the breathable backing comprises a polyester/polyether copolymer film.

14. The package of claim 12, wherein the hydrogel further comprises a preservative.

15. The package of claim 12, wherein the local anesthetic comprises a sodium-channel blocker, an antidepressant, an NMDA receptor antagonist, or an opioid, or a pharmaceutically acceptable salt thereof or a mixture thereof.

16. The package of claim 15, wherein the sodium-channel blocker is lidocaine or a pharmaceutically acceptable salt thereof.

17. The package of claim 15, wherein the antidepressant is a tricyclic antidepressant or a pharmaceutically acceptable salt thereof.

18. The package of claim 15, wherein the antidepressant is amitriptyline or a pharmaceutically acceptable salt thereof.

19. The package of claim 15, wherein the NMDA-receptor antagonist is a non-competitive NMDA-receptor antagonist or a pharmaceutically acceptable salt thereof.

20. The package of claim 15, wherein the NMDA-receptor antagonist is ketamine or a pharmaceutically acceptable salt thereof.

21. The package of claim 15, wherein the opioid is morphine or a pharmaceutically acceptable salt thereof.

22. A method of inducing local anesthesia in a mammal comprising topically applying a patch to the mammal, the patch comprising a breathable backing coated with a polyvinylpyrrolidone-based hydrogel, the hydrogel comprising one or more local anesthetics or a pharmaceutically acceptable salt thereof.

23. The method of claim 22, wherein the patch is sterile.

24. The method of claim 22, wherein the breathable backing comprises a polyester/polyether copolymer film.

25. The method of claim 22, wherein the hydrogel further comprises a preservative.

26. The method of claim 22, wherein the local anesthetic comprises a sodium-channel blocker, an antidepressant, an NMDA receptor antagonist, or an opioid, or a pharmaceutically acceptable salt thereof or a mixture thereof.

27. The method of claim 26, wherein the sodium-channel blocker is lidocaine or a pharmaceutically acceptable salt thereof.

28. The method of claim 26, wherein the antidepressant is a tricyclic antidepressant or a pharmaceutically acceptable salt thereof.

29. The method of claim 26, wherein the antidepressant is amitriptyline or a pharmaceutically acceptable salt thereof.

30. The method of claim 26, wherein the NMDA-receptor antagonist is a non-competitive NMDA-receptor antagonist or a pharmaceutically acceptable salt thereof.

31. The method of claim 26, wherein the NMDA-receptor antagonist is ketamine or a pharmaceutically acceptable salt thereof.

32. The method of claim 26, wherein the opioid is morphine or a pharmaceutically acceptable salt thereof.

33. A method of treating the pain associated with a non-intact skin indication in a mammal comprising topically applying a sterile patch to the non-intact skin indication, the patch comprising a breathable backing coated with a polyvinylpyrrolidone-based hydrogel, the hydrogel comprising one or more local anesthetics or a pharmaceutically acceptable salt thereof.

34. The method of claim 33, wherein the non-intact skin indication is a wound or burn.

35. The method of claim 33, wherein the breathable backing comprises a polyester/polyether copolymer film.

36. The method of claim 33, wherein the hydrogel further comprises a preservative.

37. The method of claim 33, wherein the local anesthetic comprises a sodium-channel blocker, an antidepressant, an NMDA receptor antagonist, or an opioid, or a pharmaceutically acceptable salt thereof or a mixture thereof.

38. The method of claim 37, wherein the sodium-channel blocker is lidocaine or a pharmaceutically acceptable salt thereof.

39. The method of claim 37, wherein the antidepressant is a tricyclic antidepressant or a pharmaceutically acceptable salt thereof.

40. The method of claim 37, wherein the antidepressant is amitriptyline or a pharmaceutically acceptable salt thereof.

41. The method of claim 37, wherein the NMDA-receptor antagonist is a non-competitive NMDA-receptor antagonist or a pharmaceutically acceptable salt thereof.

42. The method of claim 37, wherein the NMDA-receptor antagonist is ketamine or a pharmaceutically acceptable salt thereof.

43. The method of claim 37, wherein the opioid is morphine or a pharmaceutically acceptable salt thereof.

44. A polyvinylpyrrolidone-based hydrogel comprising one or more local anesthetics or a pharmaceutically acceptable salt thereof.

45. The polyvinylpyrrolidone-based hydrogel of claim 44 in sterile form.

46. The polyvinylpyrrolidone-based hydrogel of claim 44, further comprising a preservative.

47. The polyvinylpyrrolidone-based hydrogel of claim 44, wherein the local anesthetic comprises a sodium-channel blocker, an antidepressant, an NMDA receptor antagonist, or an opioid, or a pharmaceutically acceptable salt thereof or a mixture thereof.

48. The polyvinylpyrrolidone-based hydrogel of claim 47, wherein the sodium-channel blocker is lidocaine or a pharmaceutically acceptable salt thereof.

49. The polyvinylpyrrolidone-based hydrogel of claim 47, wherein the antidepressant is a tricyclic antidepressant or a pharmaceutically acceptable salt thereof.

50. The polyvinylpyrrolidone-based hydrogel of claim 47, wherein the antidepressant is amitriptyline or a pharmaceutically acceptable salt thereof.

51. The polyvinylpyrrolidone-based hydrogel of claim 47, wherein the NMDA-receptor antagonist is a non-competitive NMDA-receptor antagonist or a pharmaceutically acceptable salt thereof.

52. The polyvinylpyrrolidone-based hydrogel of claim 47, wherein the NMDA-receptor antagonist is ketamine or a pharmaceutically acceptable salt thereof.

53. The polyvinylpyrrolidone-based hydrogel of claim 47, wherein the opioid is morphine or a pharmaceutically acceptable salt thereof.
